(19) United States
(12) Patent Application Publication
(10) Pub. No.: US 2017/0336301 A1
(43) Pub. Date: Nov. 23, 2017

(54) Needle-free, safety biological sample collection system

(71) Applicant: Infinite Medical Technology Corp., Carson, CA (US)

(72) Inventor: Peter A.K. YONG, Carson, CA (US)

(21) Appl. No.: 15/371,224

(22) Filed: Dec. 7, 2016

Related U.S. Application Data
(63) Continuation of application No. 14/020,827, filed on Sep. 7, 2013, now abandoned.
Application No. 14/020,827 is a continuation of application No. 12/947,786, filed on Nov. 16, 2010, now abandoned.
Application No. 12/947,786 is a continuation-in-part of application No. 11/505,770, filed on Aug. 16, 2006, now abandoned.
Application No. 11/505,770 is a continuation-in-part of application No. 11/191,300, filed on Jul. 28, 2005, now abandoned.
(60) Provisional application No. 61/261,512, filed on Nov. 16, 2009.

(51) Int. Cl. G01N 1/12; B01L 3/00; A61B 10/00; A61B 17/00; B01L 3/02

(52) U.S. Cl. CPC G01N 1/12; B01L 3/502; A61B 10/0045; B01L 2300/0681; B01L 2300/049; B01L 2300/046; B01L 2200/026; B01L 3/5082; B01L 3/0272; B01L 3/021; A61B 2017/0046; A61B 2017/00424; A61B 2010/008; A61B 10/0096; A61B 10/007; A61B 10/0051; B01L 2400/0478; B01L 2400/0605; B01L 2400/0481

(57) ABSTRACT

A collection container for biological samples is described. In one embodiment, the collection container includes a receptacle and a lid with a sampling port. The sampling port is coupled to an aspiration tube and allows access to the contents of the receptacle. A needle-free fitting allows a mating device to be coupled to the sampling port. A valve automatically closes to prevent the sample from escaping through the sampling port when a mating device is not coupled.

### CROSS-REFERENCE TO RELATED APPLICATIONS

This application is a continuation of U.S. patent application Ser. No. 14/020,827, filed Sep. 7, 2013, which is a continuation of U.S. patent application Ser. No. 12/947,786, filed Nov. 16, 2010, now abandoned, which claims the benefit of U.S. provisional application No. 61/261,512, filed Nov. 16, 2009, and is a continuation-in-part of U.S. patent application Ser. No. 11/505,770, filed Aug. 16, 2006, now abandoned, which is a continuation-in-part of U.S. patent application Ser. No. 11/191,300, filed Jul. 28, 2005, now abandoned. The applications listed above are hereby incorporated by reference in their entireties.

### BACKGROUND

Collection containers are given to patients to collect urine, blood, and other liquid samples.

One type of collection container includes a cup and a lid. The lid has a sampling opening or a simple hole with a hinged cover that provides access to the contents of the cup without having to remove the lid. This opening may allow the sample to accidentally splash out or otherwise escape from the opening when its cover is not securely fastened. To obtain some of the sample, a tube or other elongate instrument is extended through the opening directly into the liquid sample contents. Obtaining the sample in this manner may result in drips, or otherwise create exposure to a potentially hazardous sample.

Another type of collection container uses a tube holder with an exposed needle. This tube holder makes it more difficult for the sample to splash out. However, this tube holder creates the risk of the patient or other user accidentally pricking themselves with the needle, even if the needle is recessed in the tube holder. To obtain a portion of the sample, a vacuum sample collection tube is inserted in the tube holder and used with the needle. Obtaining the sample with this port requires a vacuum sample collection tube that has a limited shelf life and loses its vacuum with time.

Patients routinely set down the lid on an unclean surface after they have removed the lid from the cup and before they fill the cup. When the lid is put back onto the cup, the exposed aspiration tube will contaminate the sample contents collected.

What is needed is a collection container which does not allow the sample to splash out, even when the sample is being transferred or the collection container is being transported with abusive handling.

What is also needed is a collection container that does not create the risk of a patient or other user accidentally pricking themselves with an exposed needle.

What is also needed is a collection container with a lid that will not contaminate the sample after the lid is placed on an unclean surface.

### SUMMARY

A collection container for a liquid sample comprises a receptacle having an open top and a lid removably coupled to the top of the receptacle. The collection container also comprises a sampling port formed in the lid. The sampling port includes an opening. The collection container also comprises an aspiration tube coupled to the opening of the sampling port. The aspiration tube reaches at least near a bottom of the receptacle. The collection container also comprises a valve coupled to a top end of the aspiration tube. The valve has a closed configuration which prevents the liquid sample from escaping through the aspiration tube. The valve also has an open configuration which allows the liquid sample to be transferred through the aspiration tube when a mating device is coupled to the valve. The collection container also comprises a fitting coupled to a top end of the valve. The fitting is capable of being removably coupled to the mating device.

### BRIEF DESCRIPTION OF THE DRAWINGS

FIGS. 1A-1C show one embodiment of a collection container 100.

FIGS. 2A-2H show one method of using collection container 100.

FIGS. 3A-3B show additional embodiments of collection container 200 and 300.

### DESCRIPTION

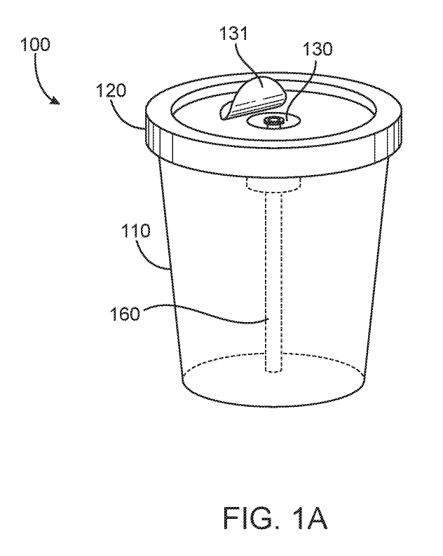

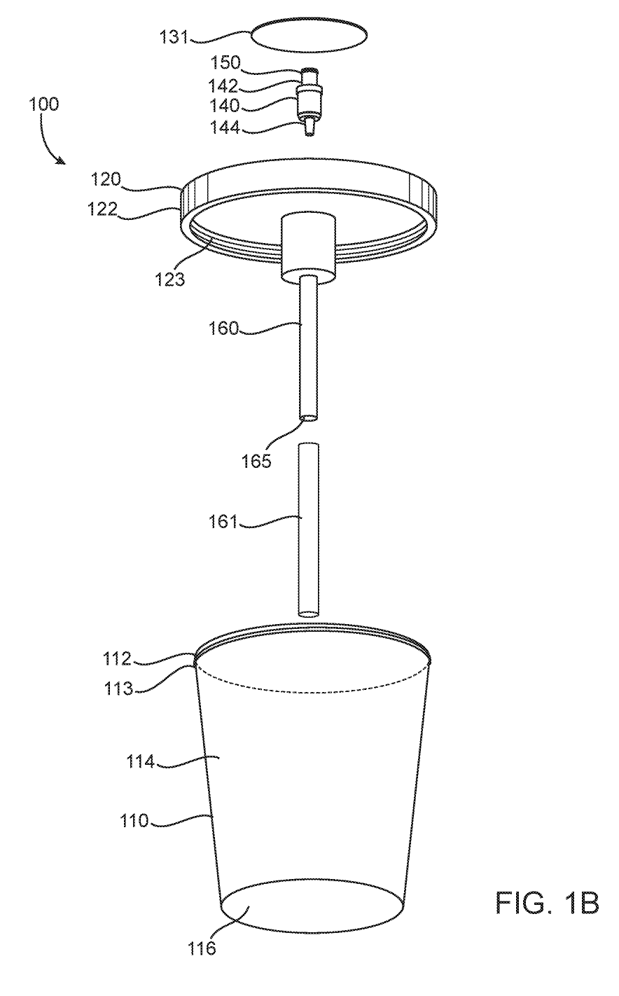

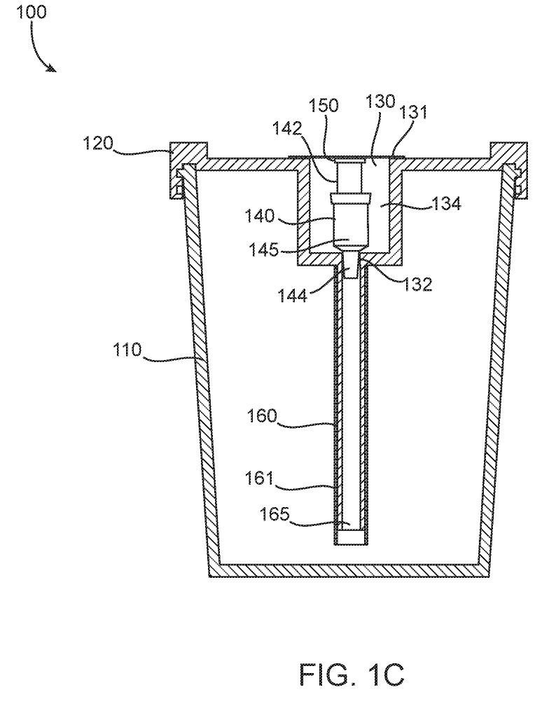

FIGS. 1A-1C show one embodiment of a collection container 100. FIG. 1A shows a perspective view of collection container 100. FIG. 1B shows an exploded view of collection container 100. FIG. 1C shows a cross-sectional view of collection container 100.

Collection container 100 includes a receptacle 110, a lid 120, a sampling port 130, a valve 140, a fitting 150, and an aspiration tube 160.

Receptacle 110 receives urine, blood, saliva, or other liquid samples. Receptacle 110 includes a mouth 112, sides 114, and a bottom 116. Mouth 112 may include threads 113 for coupling to lid 120. Sides 114 may be tapered, with mouth 112 being larger and bottom 116 being smaller. Bottom 116 may be configured to fit with a lid 120 of another receptacle 110 to allow multiple collection containers 100 to be stacked for storage or transportation. Receptacle 110 may be made of a plastic or other substantially rigid material. Receptacle 110 may be made of a material that is at least partially translucent. Receptacle 110 may be circular, oval, rectangular, or any other suitable shape.

Lid 120 seals mouth 112 of receptacle 110, and prevents the liquid sample from escaping between lid 120 and receptacle 110. Lid 120 is removably coupled to receptacle 110. Lid 120 may include a lip 122. Lip 122 may include threads 123 which are configured to couple to threads 113 of mouth 112. Alternatively, lid 120 may be coupled to receptacle with a press fit, a twist lock, or any other suitable method or device. Lip 122 may be raised and configured to receive a bottom 116 of another receptacle 110 to allow multiple collection containers 100 to be stacked for storage or transportation. Lip 122 may be knurled or textured to enhance grip.

Sampling port 130 allows access to the contents of receptacle 110 when lid 120 is coupled to receptacle 110. Sampling port 130 includes an opening 132 formed in lid 120. Sampling port 130 may also include a recess 134 formed in lid 120, with opening 132 located at a bottom of recess 134.

Sampling port 130 may be covered by a port seal 131 which keeps sampling port 130 clean until it is ready to be used. Port seal 131 may be made of paper, plastic, foil, or other suitable material. Port seal 131 may be affixed with an adhesive or other suitable method. Port seal 131 may have a pull tab, and is easily removed when sampling port 130 is ready to be used.

Valve 140 is coupled to opening 132 of sampling port 130. Valve 140 may be seated at a bottom of recess 134. Valve 140 has a top end 142, a bottom end 144, and a lumen 145. Top end 142 of valve 140 may be flush with a top of lid 120. Alternatively, top end 142 of valve 140 may be recessed with respect to the top of lid 120, or protrude above the top of lid 120.

Fitting 150 may be coupled to top end 142 of valve 140. Fitting 150 may be a luer attachment or other suitable fitting. Fitting 150 may be configured to be coupled to a sample collection device, such as any of the embodiments of a handle as described in U.S. Patent Application Publication No. 2007/0025886.

Valve 140 automatically closes to prevent the contents of receptacle 110 from leaking or escaping through sampling port 130 when lid 120 is in place. Valve 140 opens when a sample collection device is coupled to fitting 150. Valve 140 may be a uni-directional or bi-directional luer-activated checkvalve such as those available from Qosina Corporation, Edgewood, N.Y. or Baxter Healthcare Corporation, Round Lake, Ill.

Aspiration tube 160 is coupled to opening 132 of sampling port 130. Aspiration tube 160 includes a lumen 165 that is in fluid communication with lumen 145 of valve 140. When lid 120 is coupled to receptacle 110, aspiration tube 160 is of sufficient length to reach at least near bottom 116 of receptacle 110. Aspiration tube 160 allows most or all of the contents of receptacle 110 to be drawn out through aspiration tube 160.

Aspiration tube 160 may be covered by a sleeve 161 which keeps aspiration tube 160 clean or sterile until receptacle 110 is used. Sleeve 161 may be made of paper, plastic, or other suitable material. Sleeve 161 is configured to cover aspiration tube 160 and stay in place until removed. Sleeve 161 may be slightly longer than aspiration tube 160. Sleeve 161 may be open-ended or closed-ended.

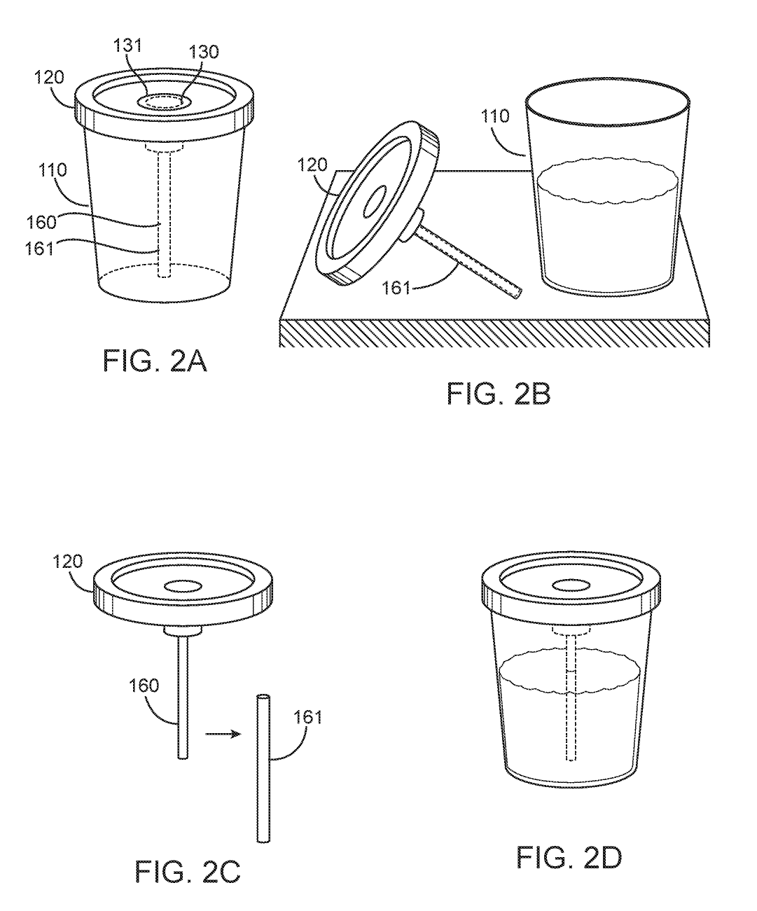

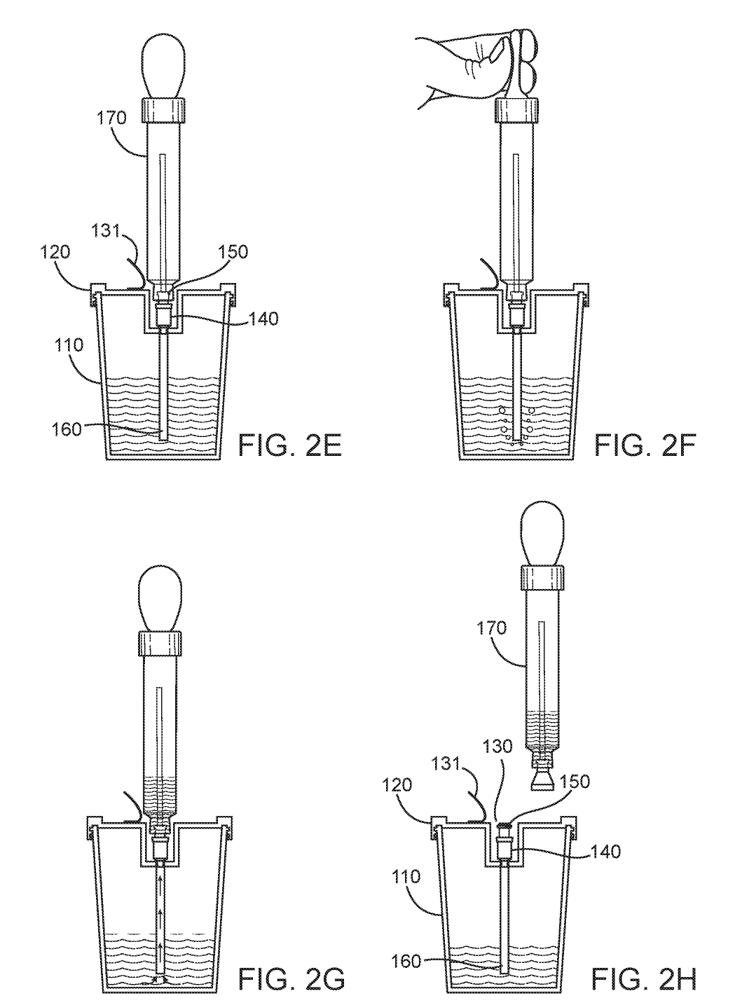

FIGS. 2A-2H show one method of using collection container 100.

FIG. 2A shows collection container 100 with receptacle 110 closed with lid 120. Collection container 100 is given to a patient.

FIG. 2B shows collection container 100 after the patient has removed lid 120 from receptacle 110. If the patient sets down lid 120, aspiration tube 160 is kept clean by sleeve 161. Receptacle 110 receives a sample from the patient.

FIG. 2C shows lid 120 held up and sleeve 161 removed from aspiration tube 160.

FIG. 2D shows lid 120 coupled back to receptacle 110, sealing the sample inside. The sample is prevented from leaking or escaping through sampling port 130 by valve 140.

FIG. 2E shows port seal 131 peeled back to expose sampling port 130, and a sample collection device 170 coupled to fitting 150 of valve 140. Valve 140 is opened when sample collection device 170 is coupled.

FIGS. 2F-2G shows sample collection device 170 being squeezed and released to create a vacuum that will draw the sample through aspiration tube 160 and valve 140 and into sample collection device 170. Sample collection device 170 may be squeezed one or more times to draw the desired amount of sample into sample collection device 170.

FIG. 2H shows sample collection device 170 disconnected from valve 140. Valve 140 is closed when sample collection device 170 is uncoupled. One or more additional sample collection devices 170 may be used to collect additional amounts of sample.

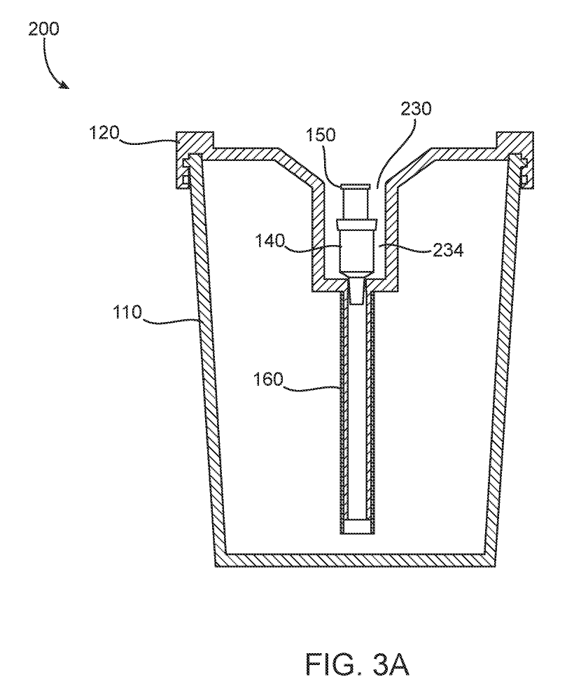

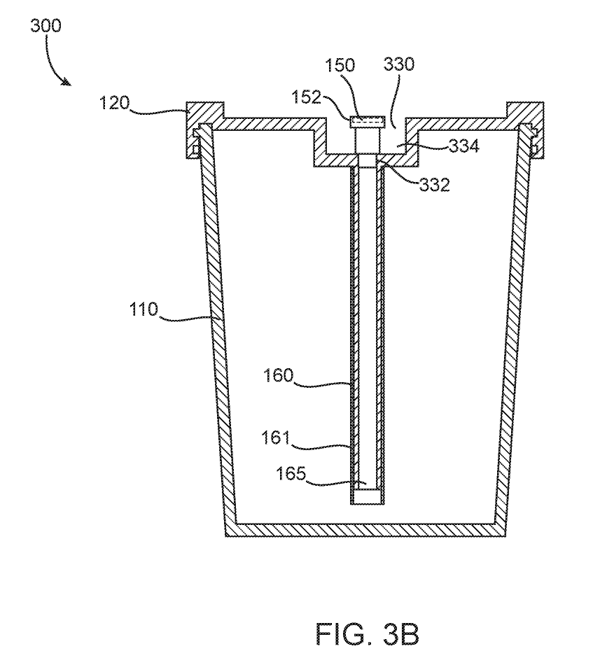

FIGS. 3A-3B show additional embodiments of collection container 200.

FIG. 3A shows another embodiment of collection container 200. Collection container 200 includes a receptacle 110, a lid 120, a sampling port 230, a valve 140, a fitting 150, and an aspiration tube 160. Collection container 200 is similar to collection container 100, but includes a sampling port 230 with a recess 234 that is shaped to guide a sample collection container to fitting 150 of valve 140.

FIG. 3B shows yet another embodiment of collection container 300. Collection container 300 includes a receptacle 110, a lid 120, a sampling port 330, a fitting 150, and an aspiration tube 160. Collection container 300 is similar to collection container 100, but does not include a valve. Fitting 150 is coupled to opening 332 in recess 334 of sampling port 330. Fitting 150 may be covered by a cap 152 which prevents the sample from escaping through sampling port 330.

While the foregoing has been with reference to particular embodiments of the invention, it will be appreciated by those skilled in the art that changes in these embodiments may be made without departing from the principles and spirit of the invention.

All publications and patent applications mentioned in this specification are herein incorporated by reference to the same extent as if each individual publication or patent application was specifically and individually indicated to be incorporated by reference.

What is claimed is:

1. A collection container for a liquid sample, the collection container comprising:
a receptacle having an open top;
a lid removably coupled to the top of the receptacle;
a sampling port formed in the lid, the sampling port including an opening;
an aspiration tube coupled to the opening of the sampling port, the aspiration tube reaching at least near a bottom of the receptacle;
a valve coupled to a top end of the aspiration tube, the valve having a closed configuration which prevents the liquid sample from escaping through the aspiration tube, the valve having an open configuration which allows the liquid sample to be transferred through the aspiration tube when a mating device is coupled to the valve; and
a fitting coupled to a top end of the valve, the fitting capable of being removably coupled to the mating device.

2. The collection container of claim 1, wherein the sampling port includes a recess formed in the lid.

3. The collection container of claim 1, wherein the valve is a luer activated checkvalve.

4. The collection container of claim 1, wherein the fitting is a needle-free connector.

5. The collection container of claim 1, wherein the fitting is a luer connector.

6. The collection container of claim 1, wherein the fitting does not protrude from a top of the lid.

7. The collection container of claim 1, further comprising:
a sleeve removably coupled to the aspiration tube, the sleeve covering the aspiration tube.

8. The collection container of claim 7, wherein the sleeve extends past a bottom of the aspiration tube.

9. A collection container for a liquid sample, the collection container comprising:
a receptacle having an open top;
a lid removably coupled to the top of the receptacle;
a sampling port formed in the lid, the sampling port including an opening;
an aspiration tube coupled to the opening of the sampling port, the aspiration tube reaching at least near a bottom of the receptacle; and
a means for removably coupling to a mating device, the means for removably coupling being coupled to a top of the aspiration tube.

10. The collection container of claim 9, wherein the sampling port includes a recess formed in the lid.

11. The collection container of claim 9, wherein the means for removably coupling is needle-free.

12. The collection container of claim 9, wherein the means for removably coupling does not protrude from a top of the lid.

13. The collection container of claim 9, further comprising:
a sleeve removably coupled to the aspiration tube, the sleeve covering the aspiration tube.

14. The collection container of claim 13, wherein the sleeve extends past a bottom of the aspiration tube.

15. The collection container of claim 9, further comprising:
a means for automatically closing the opening of the sampling port when a mating device is not coupled to the means for removably coupling.

16. A method for collecting a liquid sample, the method comprising:
providing a receptacle having an open top;
removing a lid removably coupled to the top of the receptacle;
placing the lid on a surface;
depositing the liquid sample into the receptacle;
picking the lid up from the surface;
removing a sleeve from an aspiration tube coupled to an opening of a sampling port formed in the lid; and
coupling the lid to the top of the receptacle to seal the receptacle.

17. The method of claim 16, further comprising:
coupling a mating device to a fitting coupled to a top of the aspiration tube; and
using the mating device to transfer a portion of the liquid sample through the aspiration tube into the mating device.
